(19) United States
(12) Patent Application Publication
(10) Pub. No.: US 2026/0165916 A1
(43) Pub. Date: Jun. 18, 2026

(54) BIPHASIC MAKEUP REMOVAL COSMETIC COMPOSITION

(71) Applicant: L'OREAL, Paris (FR)

(72) Inventors: Vanessa DUFFET, Chevilly la Rue (FR); Julie DEVILLECHABROLLE, Chevilly la Rue (FR)

(73) Assignee: L'OREAL, Paris (FR)

(21) Appl. No.: 19/123,886

(22) PCT Filed: Nov. 7, 2023
(86) PCT No.: PCT/EP2023/080989

§ 371 Date: Apr. 24, 2025

(30) Foreign Application Priority Data
Nov. 8, 2022 (FR) FR2211630

(51) Int. Cl. A61K 8/03; A61K 8/31; A61K 8/37; A61Q 1/14

(52) U.S. Cl. CPC A61K 8/03; A61K 8/31; A61K 8/37; A61Q 1/14; A61K 2800/30

(57) ABSTRACT

The present invention relates to a cosmetic composition consisting of an aqueous phase and an oily phase which are separate, wherein the oily phase comprises:
from 48% to 82% by weight of isododecane with respect to the total weight of oily phase;
from 10% to 22% by weight of isopropyl palmitate with respect to the total weight of oily phase; and
from 8% to 18% by weight of isopropyl myristate with respect to the total weight of oily phase,
the composition not containing C15-C19 alkanes.

It also relates to a specific ternary mixture, and associated methods and cosmetic uses.

The present invention relates to a biphasic composition consisting of an aqueous phase and an oily phase which are separate, the oily phase comprising specific oils. The present invention also relates to the use of such a composition, as well as to a makeup removal method using such a composition.

Compositions consisting of two separate phases, particularly an aqueous phase and an oily phase which are separate and are not emulsified in each other at rest, are generally referred to by the term “biphasic composition.” They differ from emulsions by the fact that, at rest, the two phases are separate instead of being emulsified in each other. The use of these biphasic compositions requires prior stirring in order to form an emulsion, the latter needing to be of sufficient quality and stability to allow for a homogeneous application of both phases on the skin or the keratin material to which it is applied. At rest, said phases must separate quickly from each other and return to their initial state, this phenomenon being more commonly known by the term “phase separation” or “demixing.”

Relatively fast demixing of the two phases after their use is one of the desired qualities of biphasic compositions. Indeed, achieving a rapid phase separation is desirable for various reasons, particularly because a poor separation of the two phases is perceived as unsightly by users and may cause the product to become “triphasic” as it is used over time.

Furthermore, marketed biphasic compositions are in particular used for removing makeup from the face, in particular from the eyes, and contain cyclopentasiloxane.

However, the cosmetics market tends to be focused on compositions that are increasingly natural, comprising more environmentally-friendly compounds and/or originating from biomass. In particular, there is a need for compositions free from cyclopentasiloxane.

Consequently, there remains a need for novel stable biphasic compositions, consisting of two separate, non-mixable phases, free from cyclopentasiloxane, and which have good demixing qualities, while ensuring effective makeup removal. There is also a need for natural compositions. “Natural composition” means a cosmetic composition mainly formulated with natural ingredients and/or ingredients of natural origin. “Natural ingredient” means a compound that is obtained directly from the ground or soils or from plants or animals, if applicable, by means of one or more physical processes such as, for example, grinding, refining, distillation, purification, or filtration. Ingredients “of natural origin” means natural compounds having undergone one or more chemical treatments or related industrial treatments bringing about modifications that are not detrimental to the essential qualities of the compound, and/or a compound mainly comprising natural components which may or may not have undergone transformations as indicated above.

The inventors surprisingly discovered that the ternary association of isododecane, isopropyl palmitate and isopropyl myristate in specific proportions, coupled with an aqueous phase, makes it possible to obtain a biphasic composition that meets these needs. The compositions according to the invention are stable, free from cyclopentasiloxane, and particularly have good demixing qualities and are very effective for makeup removal.

More specifically, the present invention relates to a cosmetic composition consisting of an aqueous phase and an oily phase which are separate, characterized in that the oily phase comprises:
from 48% to 82% by weight of isododecane with respect to the total weight of oily phase;
from 10% to 22% by weight of isopropyl palmitate with respect to the total weight of oily phase; and
from 8% to 18% by weight of isopropyl myristate with respect to the total weight of oily phase, the composition not containing C15-C19 alkanes.

Such a composition according to the invention is a biphasic composition.

The present invention also relates to a composition consisting of:
from 48% to 82% of isododecane by weight with respect to the total weight of the composition;
from 10% to 22% by weight of isopropyl palmitate with respect to the total weight of the composition; and
from 8% to 18% by weight of isopropyl myristate with respect to the total weight of the composition,
the composition not containing C15-C19 alkanes.

This composition is also called a “ternary mixture” in this application. The ternary mixture corresponds to the preferred oily phase of the biphasic composition according to the invention.

The present invention also relates to a method of makeup removal and/or cleaning and/or care of the keratin materials comprising the application of a biphasic composition or a ternary mixture according to the invention on said keratin materials, in particular the keratin materials of the eyes, preferably the eyelashes.

The present invention also relates to the use of a biphasic composition or a ternary mixture according to the invention for makeup removal and/or cleaning and/or care of the keratin materials, particularly the keratin materials of the eyes, preferably the eyelashes.

“Keratin materials” refers to the skin, mucosa and/or skin appendages. Preferably the keratin materials are the skin, particularly facial skin, mucosa such as lips, and/or skin appendages such as eyelashes.

### Oily Phase

The oily phase of the biphasic composition according to the invention comprises:
from 48% to 82% by weight of isododecane with respect to the total weight of oily phase;
from 10% to 22% by weight of isopropyl palmitate with respect to the total weight of oily phase; and
from 8% to 18% by weight of isopropyl myristate with respect to the total weight of oily phase, the composition not containing C15-C19 alkanes.

The ternary mixture composition according to the invention consists of:
from 48% to 82% of isododecane by weight with respect to the total weight of the composition;
from 10% to 22% by weight of isopropyl palmitate with respect to the total weight of the composition; and
from 8% to 18% by weight of isopropyl myristate with respect to the total weight of the composition, the mixture not containing C15-C19 alkanes.

The feature “the composition not containing C15-C19 alkanes” or “the mixture not containing C15-C19 alkanes” is understood to mean that the biphasic composition or the ternary mixture, respectively, are totally free from C15-C19 alkanes. These excluded C15-C19 alkanes are particularly C15-C19 branched alkanes such as those marketed by Seppic under the name EMOGREEN L19.

Preferably, the biphasic composition and the ternary mixture according to the invention contain no alkane other than isododecane.

The biphasic composition and the ternary mixture according to the invention comprise from 48% to 82% by weight of isododecane with respect to the total weight of oily phase. Preferably, the biphasic composition and the ternary mixture according to the invention comprise from 50% to 75% by weight of isododecane with respect to the total weight of oily phase, preferably from 55% to 70% by weight, preferably from 57% to 68% by weight.

The biphasic composition and the ternary mixture according to the invention comprise from 10% to 22% by weight of isopropyl palmitate with respect to the total weight of oily phase. Preferably, the biphasic composition and the ternary mixture according to the invention comprise from 15% to 22% by weight of isopropyl palmitate with respect to the total weight of oily phase, preferably from 17% to 22% by weight.

The biphasic composition and the ternary mixture according to the invention comprise from 8% to 18% by weight of isopropyl myristate with respect to the total weight of oily phase. Preferably, the biphasic composition and the ternary mixture according to the invention comprise from 10% to 18% by weight of isopropyl myristate with respect to the total weight of oily phase, preferably from 15% to 18% by weight.

Preferably, the biphasic composition and the ternary mixture according to the invention are substantially free from cyclopentasiloxane. “Substantially free from cyclopentasiloxane” means that this composition comprises less than 1% by weight of cyclopentasiloxane with respect to the total weight of the composition, preferably less than 0.5% by weight, preferably less than 0.1% by weight. Preferably, the biphasic composition and the ternary mixture according to the invention are totally free from cyclopentasiloxane.

### Aqueous Phase

The aqueous phase of the biphasic composition comprises water.

The aqueous phase optionally contains at least one organic solvent that is soluble or miscible in water, at 25° C.

The water-soluble or water-miscible solvents suitable for the invention comprise short-chain linear or branched alkanols for example at C2-C4, such as ethanol, isopropanol, propanol or butanol; diols or polyols particularly with 2 to 20 carbon atoms, preferably 2 to 6 carbon atoms, such as glycerol, ethylene glycol, 1,2-propylene glycol, 1,3-butylene glycol, hexylene glycol, diethylene glycol, 1,3-propanediol, 1,2-pentadiol, dipropylene glycol, 2-ethoxyethanol, diethylene glycol monomethylether, triethylene glycol monomethylether, sorbitol, polyethylene glycols with 2 to 200 ethylene oxide units and mixtures thereof.

Preferably, the aqueous phase comprises at least 60% by weight water with respect to the total weight of aqueous phase, preferably at least 70% by weight, preferably a content of between 80% and 98% by weight.

The aqueous phase can further comprise conventional cosmetic additives, such as electrolytes, chelating agents, preservatives and/or surfactants.

The electrolyte may be chosen from sodium chloride or sodium sulfate. The electrolyte is preferably sodium chloride.

Preferably, the active substance content of electrolyte(s) is between 0.2% and 5% by weight with respect to the total weight of the composition, preferably between 0.5% and 4% by weight, preferably between 1% and 3% by weight.

In particular, the aqueous phase can comprise at least one surfactant chosen from anionic surfactants, amphoteric surfactants and non-ionic surfactants. Preferably, the aqueous phase comprises at least one non-ionic surfactant, preferably chosen from alkylpolyglucosides, esters of fatty acid comprising 6 to 12 carbon atoms and of polyglycerol comprising 5 to 8 glycerol units, and mixtures thereof.

Preferably, the aqueous phase comprises from 0.015% to 7%, preferably from 0.01% to 5%, and preferentially from 0.02% to 1%, with weight of surfactant with respect to the total weight of aqueous phase.

Alkylpolyglucosides (APGs) belong to the alkylpolyglycoside family and generally comply with the following structure: R(O)(G)ₓ
wherein the radical R is a C₁₂-C₂₂ linear or branched alkyl radical, G is a glucose residue and x varies from 1 to 5, preferably from 1.05 to 2.5 and more preferably from 1.1 to 2.

According to the invention, the term alkylpolyglucoside (APG) also encompasses polyalkyl(poly)glucosides. As alkyl- and polyalkyl-glucosides or polyglucosides, those containing an alkyl group comprising from 6 to 30 carbon atoms and preferably from 6 to 18, even from 8 to 16 carbon atoms are preferably used, and containing a glucoside group comprising preferably from 1 to 5, in particular 1, 2 to 3 glucoside units. The alkylpolyglucosides can be chosen for example from decylglycoside (Alkyl-C₉/C₁₁-polyglucoside (1.4)) such as the product marketed under the trade name Mydol 10® by Kao Chemicals or the product marketed under the trade name Plantacare 2000 UP® by Henkel and the product marketed under the trade name ORAMIX NS 10® by SEPPIC; caprylyl/capryl glucoside such as the product marketed under the trade name Plantacare KE 3711® by Cognis or ORAMIX CG 110® by SEPPIC; laurylglucoside such as the product marketed under the trade name Plantacare 1200 UP® by Henkel or Plantaren 1200 N® by Henkel; cocoglucoside such as the product marketed under the trade name Plantacare 818 UP® by Henkel; caprylylglucoside such as the product marketed under the trade name Plantacare 810 UPR by Cognis; and mixtures thereof. Preferably, the APG is decyl glucoside.

The fatty acid and polyglycerol ester is formed from at least one fatty acid comprising from 6 to 12 carbon atoms and polyglycerol comprising from 5 to 8 glycerol units.

The fatty acid may be saturated or unsaturated. Preferably, the fatty acid and polyglycerol ester is a mono- or diester, and preferably a mono-ester.

The term “polyglycerol” designates glyceryl polymers that are linear chains of 5 to 8 glycerol units.

The esters that are more particularly considered according to the present invention are esters resulting from the esterification of polyglycerol and carboxylic acid(s) at C8, such as caprylic acid. The carboxylic acid may be linear or branched, saturated or unsaturated. Preferably, it is a linear monocarboxylic acid.

According to one particular embodiment, esters suitable for this invention can be derived from esterification of a polyglycerol by one or several identical or different carboxylic acids. They may be a hydroxylated mono-ester, a hydroxylated di-ester, a hydroxylated tri-ester, or a mixture thereof.

In a preferred embodiment of the invention, the ester is chosen from the esters of fatty acid comprising 8 carbon atoms and of polyglycerol comprising 6 glycerol units.

The esters of fatty acid comprising 8 carbon atoms and of polyglycerol comprising 6 glycerol units is formed (i) from at least one fatty acid comprising an alkyl or alkenyl chain containing 8 carbon atoms (also called C8 fatty acid), and (ii) from 6 glycerol units.

Preferably, the C8 fatty acid is saturated, and contains a linear alkyl chain. Preferably, the C8 fatty acid is caprylic acid.

In a preferred embodiment of the invention, the ester of fatty acid comprising 8 carbon atoms and of polyglycerol comprising 6 glycerol units is polyglyceryl monocaprylate comprising 6 glycerol units, i.e. polyglyceryl-6 monocaprylate (polyglyceryl-6 caprylate).

### Biphasic Composition

The biphasic composition according to the invention consists of an aqueous phase and an oily phase which are separate.

Preferably, the weight ratio between the aqueous phase and the oily phase is between 1 and 6, preferably between 1.03 and 5.8, preferably between 1.04 and 5.7.

These ratio values make it possible to obtain a biphasic composition that is excellent at makeup removal and also has a satisfactory visual appearance.

This invention also relates to the use of the biphasic composition as defined hereinabove, or of the ternary mixture as defined hereinabove, for the makeup removal and/or the cleaning of keratin materials, preferably the skin and/or mucosa (such as the lips) and/or keratin fibers (such as the eyelashes).

The present invention also relates to a method of makeup removal and/or cleaning of keratin materials, preferably the skin and/or mucosa (such as the lips) and/or keratin fibers (such as the eyelashes), comprising the application on the keratin materials of a biphasic composition according to the invention, or of a ternary mixture according to the invention.

The expressions “between . . . and . . . ”, “ranging from . . . to . . . ” and “varies from . . . to . . . ” are to be understood to be inclusive of the limits, unless specified otherwise.

Concrete, yet non-limiting, examples, illustrating the invention, will now be provided. Unless stated otherwise, the pressure in the examples is atmospheric pressure.

### EXAMPLES

### Example 1: Preparation of Compositions According to the Invention and Comparative Compositions

### 1. Experiment Plan Conducted on 4 Variables (i.e., Oil Content):

% by weight with respect to the weight of oily phase:

Isododecane (QS)—Isopropyl palmitate (from 0 to 30%)—Isopropyl myristate (from 0 to 30%)-C15-C19 alkanes (0 to 35%)

### Oily Phase Formulation

In a beaker, add the 4 oils (variable contents) and homogenize for 20 minutes cold.

The compositions of the oily phases are indicated in the Table 2 to 5 hereinbelow. In Tables 2 to 5, the percentages are by weight with respect to the total weight of oily phase.

### Aqueous Phase Formulation

### Aqueous Phase Composition

TABLE 1

| | | Percentage (% |
|---|---|---|
| | | by weight of |
| Phase | Raw material | aqueous phase) |
| A | Water | 50% |
| A | EDTA | 0.2% |
| A | Decyl glucoside | 0.2% |
| | (Oramix NS10 from | |
| | Seppic) | |
| A | Electrolytes | 1.7% |
| A | Preservative | QS |
| B | Water | QS 100 |
| B | Hexylene glycol | 0.5% |

The aqueous phase is prepared as follows:

In a beaker, add the raw materials of phase A and heat to 65° C. After 20 minutes of dispersion, add the raw materials of B (the water must be cold) and disperse for 10 minutes.

### Integration in Biphasic Composition

### Measure the Density of the Two Phases.

To obtain a biphasic composition with an 85% aqueous phase/15% oily phase weight ratio, perform the following calculation:

$${Volume}_{( mL )} = \frac{{Ratio}_{aqueous phase}}{Densit {{e}^{′}}_{aqueous phase}} + \frac{{Ratio}_{oily phase}}{Densit {{e}^{′}}_{oily phase}} \qquad [ Math 1 ]$$

$${m}_{phase} = \frac{{Volume}_{packaging} * {Ratio}_{phase}}{{Volume}_{( mL )}} \qquad [ Math 2 ]$$

Calculate mₚₕₐₛₑ for each phase and pour the corresponding mass into the chosen packaging.

Each of the following compositions is obtained by mixing the aqueous phase from Table 1 with an oily phase as detailed hereinafter (Tables 2 to 5).

a. Height of Oily Phase (Clear) 45 Seconds after Stirring in Centimeters

Protocol: stir the biphasic composition 10 times by turning then start the timer. After 45 seconds, measure the height of the demixed oily phase using a ruler.

### Target: 0.5 Centimeters High

b. Height of Oily Phase (Clear) 1 Minute after Stirring in Centimeters

Protocol: stir the biphasic composition 10 times by turning then start the timer. After one minute, measure the height of the demixed oily phase using a ruler.

### Target: 1.2 Centimeters High

c. Presence of Droplets on Walls of Oily Phase after 4 Days of Stirring

Protocol: for 5 days, stir the biphasic composition 10 times by turning every day. Leave the biphasic composition to stand for 24 h then repeat the operation the following day. On the 5^{th} day, do not stir and evaluate the biphasic composition.

The presence of droplets on the walls of oily phase may be detected, and annotated as follows:

0=no droplets; 1=very fine/sparse droplets; 2=fine/few droplets; 3=medium/more numerous droplets; 4: large/numerous droplets

### Target: 2 or Less

d. Definition of Interface after 4 Days of Stirring

Protocol: for 5 days, stir the biphasic composition 10 times by turning every day. Leave the biphasic composition to stand for 24 h then repeat the operation the following day. On the 5^{th} day, do not stir and evaluate the biphasic composition. The interface corresponds to the separating line between the aqueous phase and the oily phase.

Instabilities are annotated as follows:

1=very cloudy interface/large droplets; 2=cloudy interface/medium droplets; 3=clearly defined interface-sparse droplets; 4=clearly defined interface-some microdroplets; 5=clearly defined interface

### Target: 3 or More

### Influence of C15-C19 Alkane Content

TABLE 2

| | | Comparative 1 | Comparative 2 |
|---|---|---|---|
| Oily phase | Example 1 | (Cp1) | (Cp2) |
| (% by weight of | according to the | outside | outside |
| oily phase) | invention | invention | invention |
| Isododecane | QS 100 | QS 100 | 0% |
| Isopropyl | 17.7% | 17.7% | 17.7% |
| myristate | | | |
| Isopropyl | 15% | 15% | 15% |
| palmitate | | | |
| C15-C19 alkanes | 0% | 0.1% | QS 100 |
| Height of oily | 0.5 cm (=target) | 0.7 cm | 1.2 cm |
| phase after 45 s of | | | |
| stirring | | | |

The demixing rate of the 2 phases is evaluated by measuring the height of the oily phase after 45 seconds of stirring. The target is placed at 0.5 cm. When C15-C19 alkanes are added to the composition (Comparative 1) or replace isododecane entirely (Comparative 2), the target is not attained.

The Biphasic Composition and the Ternary Mixture According to the Invention Therefore Must not Contain any Alkane Other than Isododecane.

### Influence of Isopropyl Myristate Content

TABLE 3

| | Ex2 | Ex3 | Ex4 | Ex5 | | |
|---|---|---|---|---|---|---|
| Oily phase | according | according | according | according | Cp3 | Cp4 |
| (% by weight | to the | to the | to the | to the | outside | outside |
| of oily phase) | invention | invention | invention | invention | invention | invention |
| Isododecane | QS 100 | QS 100 | QS 100 | QS 100 | QS 100 | QS 100 |
| Isopropyl | 8% | 12% | 15% | 18% | 7.9% | 18.1% |
| myristate | | | | | | |
| Isopropyl | 15% | 15% | 15% | 15% | 15% | 15% |
| palmitate | | | | | | |
| Height of oily | 1.2 cm | 1.2 cm | 1.25 cm | 1.25 cm | 1.4 cm | 1.3 cm |
| phase after 1 | (=target) | (=target) | (=target) | (=target) | | |
| min of stirring | | | | | | |
| Presence of | 1 | 1 | 1.1 | 1.2 | 2.2 | 3 |
| droplets on | (=target) | (=target) | (=target) | (=target) | | |
| walls of oily | | | | | | |
| phase at D 4 | | | | | | |
| of stirring | | | | | | |

The height of the oily phase after 1 minute of stirring and the presence of droplets on the walls after 4 days of stirring were evaluated.

When the isopropyl myristate content is strictly less than 8% or strictly greater than 18%, the targets are not attained.

Therefore, Isopropyl Myristate Must be Present from 8 to 18% by Weight with Respect to the Total Weight of Oily Phase.

### Influence of Isopropyl Palmitate Content

TABLE 4

| | Ex6 | Ex7 | Ex8 | Ex9 | | |
|---|---|---|---|---|---|---|
| Oily phase | according | according | according | according | Cp5 | Cp6 |
| (% by weight | to the | to the | to the | to the | outside | outside |
| of oily phase) | invention | invention | invention | invention | invention | invention |
| Isododecane | QS 100 | QS 100 | QS 100 | QS 100 | QS 100 | QS 100 |
| Isopropyl | 18% | 18% | 18% | 18% | 18% | 18% |
| myristate | | | | | | |
| Isopropyl | 10% | 14% | 18% | 22% | 9.9% | 22.1% |
| palmitate | | | | | | |
| Definition of | 3.8 | 4.8 | 5.3 | 3.8 | 3.7 | 2.4 |
| interface at | (=target) | (=target) | (=target) | (=target) | (=target) | |
| D 4 of stirring | | | | | | |
| Presence of | 1.9 | 1.6 | 1.6 | 1.6 | 2.6 | 2.7 |
| droplets on | (=target) | (=target) | (=target) | (=target) | | |
| walls of oily | | | | | | |
| phase at D 4 | | | | | | |
| of stirring | | | | | | |

The definition of the interface after 4 days of stirring and the presence of droplets on the walls after 4 days of stirring were evaluated. When the isopropyl palmitate content is less than 10%, the target is not attained for droplet presence. When the content is greater than 22%, the 2 targets are not attained.

Therefore, Isopropyl Palmitate Must be Present from 10 to 22% by Weight with Respect to the Total Weight of Oily Phase.

### Influence of Isododecane Content

TABLE 5

| | Ex10 | Ex11 | Ex12 | Ex13 | | |
|---|---|---|---|---|---|---|
| Oily phase | according | according | according | according | Cp7 | Cp8 |
| (% by weight | to the | to the | to the | to the | outside | outside |
| of oily phase) | invention | invention | invention | invention | invention | invention |
| Isododecane | 48% | 60% | 71% | 82% | 47.9% | 82.1% |
| Isopropyl | 37% | 25% | 14% | 3% | 37.1% | 2.9% |
| myristate | | | | | | |
| Isopropyl | 15% | 15% | 15% | 15% | 15% | 15% |
| palmitate | | | | | | |
| Height of oily | 0.6 cm | 0.5 cm | 0.5 cm | 0.4 cm | 0.8 cm | 0.4 cm |
| phase after | (=target) | (=target) | (=target) | (=target) | | (=target) |
| 45 s of stirring | | | | | | |
| Presence of | 1.9 | 1.8 | 1.6 | 1.9 | 2.7 | 2.6 |
| droplets on | (=target) | (=target) | (=target) | (=target) | | |
| walls of oily | | | | | | |
| phase at D 4 | | | | | | |
| of stirring | | | | | | |

The height of the oily phase after 45 seconds of stirring and the presence of droplets on the walls after 4 days of stirring were evaluated. When the isododecane content is less than 48%, the 2 targets are not attained. When the content is greater than 82%, the target is not attained for droplet presence.

Therefore, Isododecane Must be Present from 48 to 82% by Weight with Respect to the Total Weight of Oily Phase.

Isopropyl palmitate is essential particularly to obtain few droplets after 4 days of stirring. Isopropyl myristate is essential particularly to obtain good demixing.

### 2. Influence of Ratios

Other ratios were tested with the following compositions (same oily phase composition, see hereinafter):
70% aqueous phase/30% oily phase ratio; and
56% aqueous phase/44% oily phase ratio.

### Aqueous Phase Composition

The aqueous phase has the same composition as that of Table 1.

### Oily Phase Composition

TABLE 6

| Phase | Raw material | Percentage |
|---|---|---|
| A | Isododecane | 65% |
| A | Isopropyl palmitate | 15% |
| A | Isopropyl myristate | 17.7% |
| A | Additives | QS 100 |

### Tests at Different Ratios

TABLE 7

| | Ratio 1 | Ratio 2 | Ratio 3 |
|---|---|---|---|
| Percentage | 85 | 70 | 56 |
| aqueous phase | | | |
| Percentage | 15 | 30 | 44 |
| oily phase | | | |
| Presence of | Fine and sparse | Fine and sparse | Fine and sparse |
| droplets on | (=target) | (=target) | (=target) |
| walls of oily | | | |
| phase at D 4 | | | |
| of stirring | | | |
| Definition of | Clearly defined, | Clearly defined, | Clearly defined, |
| interface at | no droplets | no droplets | no droplets |
| D 4 of stirring | (=target) | (=target) | (=target) |

On the same phase composition but different ratios, the tests are all stable with respect to the targets.

### Example 2: Evaluation of Compositions According to the Invention and Comparative Compositions

The makeup removal effectiveness of different biphasic compositions (reference, Invention 1 or 1′ and Invention 2 or 2′) is evaluated, on a foundation and a mascara:

Foundation (FDT) application: 0.1 mL of Maybelline Fit Me Matte Pore less color 220 for each half-face.

Mascara application: 30 passes of Voluminous Blackest Black mascara on the upper eyelashes.

Wait 30 minutes before makeup removal:

For the FDT: for each half-face, use 5 cottons soaked in 2 ml of biphasic composition are used. Remove makeup from each part of the face. Wait 5 minutes, then apply 2 ml of Lancôme Bifacil on a cotton, with a maximum of 6 cottons.

Count the number of cottons soaked in Bifacil required to remove the residue.

For the mascara: for each eye, use 3 cottons soaked in 2 ml of biphasic composition. Remove makeup from the eye. Wait 5 minutes, then apply 2 ml of Bifacil on a cotton, with a maximum of 6 cottons.

Count the number of cottons soaked in Bifacil required to remove the residue.

### 1/Formulas Tested (Ratio 85:15):

### Total Phase Composition+Phase Ratio

TABLE 8

| | Reference (Garnier | | |
|---|---|---|---|
| | SkinActive Biphase | | |
| | Micellar All-in-1) | Invention 1 | Invention 2 |
| Aqueous | Composition of Table 1 | | Composition of |
| phase | | | Table 1 + 0.03% |
| (85%) | | | polyglyceryl-6 |
| | | | caprylate |

| | | |
|---|---|---|
| Oily | By weight of total | By weight of total weight of oily |
| phase | weight of oily phase: | phase: Isododecane (65%) |
| (15%) | Cyclopentasiloxane | Isopropyl palmitate (15%) |
| | (60%) | Isopropyl myristate (17.7%) |
| | Isohexadecane | Additives QS 100 |
| | (40%) | |

### Results

The reference is at 1 makeup removal cotton for the FDT and 1.5 cottons for the mascara, whereas the 2 biphasic compositions according to the invention are at 0 cottons for the FDT and 1 cotton for the mascara.

The 2 biphasic compositions according to the invention remove makeup better than the reference.

### 2/Formulas Tested (Ratio 70:30):

### Total Phase Composition+Phase Ratio

TABLE 9

| | Reference (Garnier | | |
|---|---|---|---|
| | CleanSensitive 2- | | |
| | in-1 Express | | |
| | face + eye makeup | | |
| | remover with no | | |
| | rubbing) | Invention 1′ | Invention 2′ |
| Aqueous | Composition of Table 1 | | Composition of |
| phase | | | Table 1 + 0.03% |
| (70%) | | | polyglyceryl-6 |
| | | | caprylate |

| Oily | By weight of total | By weight of total weight of |
|---|---|---|
| phase | weight of oily phase: | oily phase: Isododecane (65%) |
| (30%) | Cyclopentasiloxane | Isopropyl palmitate (15%) |
| | (60%) | Isopropyl myristate (17.7%) |
| | Isohexadecane | Additives QS 100 |
| | (40%) | |

### Results

The reference is at 0 makeup removal cottons for the FDT and 0.5 cottons for the mascara, whereas the 2 biphasic compositions according to the invention are at 0 cottons for the FDT and 0 cottons for the mascara.

The 2 biphasic compositions according to the invention remove makeup better than the reference.

1. A cosmetic composition comprising an aqueous phase and an oily phase which are separate, characterized in that the oily phase comprises:
from 48% to 82% by weight of isododecane with respect to the total weight of the oily phase;
from 10% to 22% by weight of isopropyl palmitate with respect to the total weight of the oily phase; and
from 8% to 18% by weight of isopropyl myristate with respect to the total weight of the oily phase,
the composition not containing C15-C19 alkanes.

2. A cosmetic consisting of:
from 48% to 82% of isododecane by weight with respect to the total weight of the composition;
from 10% to 22% by weight of isopropyl palmitate with respect to the total weight of the composition; and
from 8% to 18% by weight of isopropyl myristate with respect to the total weight of the composition,
the composition not containing C15-C19 alkanes.

3. The cosmetic according to claim 1, which comprises from 50% to 75% by weight of isododecane with respect to the total weight of the oily phase.

4. The cosmetic according to claim 1, which comprises from 15% to 22% by weight of isopropyl palmitate with respect to the total weight of the oily phase, preferably from 17% to 22% by weight.

5. The cosmetic according to claim 1, which comprises from 10% to 18% by weight of isopropyl myristate with respect to the total weight of the oily phase.

6. The cosmetic according to claim 1, which is substantially free from cyclopentasiloxane, preferably totally free from cyclopentasiloxane.

7. The composition according to claim 1, wherein the aqueous phase comprises water and optionally at least one water-solution or miscible solvent, at 25° C.

8. The composition according to claim 1, wherein the aqueous phase comprises at least 60% by weight water with respect to the total weight of the aqueous phase.

9. The composition according to claim 1, wherein the aqueous phase comprises at least one surfactant chosen from anionic surfactants, amphoteric surfactants and non-ionic surfactants.

10. The composition according to claim 1, wherein the weight ratio between the aqueous phase and the oily phase is between 1 and 6.

11. A method of makeup removal and/or cleaning and/or care of keratin materials comprising the application of a composition according to claim 1 on said keratin materials.

12. (canceled)

13. The cosmetic according to claim 2, which comprises from 50% to 75% by weight of isododecane with respect to the total weight of an oily phase.

14. The cosmetic according to claim 2, which comprises from 15% to 22% by weight of isopropyl palmitate with respect to the total weight of an oily phase.

15. The cosmetic according to claim 3, which comprises from 15% to 22% by weight of isopropyl palmitate with respect to the total weight of an oily phase.

16. The cosmetic according to claim 2, which comprises from 10% to 18% by weight of isopropyl myristate with respect to the total weight of an oily phase.

17. The cosmetic according to claim 3, which comprises from 10% to 18% by weight of isopropyl myristate with respect to the total weight of an oily phase.

18. The cosmetic according to claim 4, which comprises from 10% to 18% by weight of isopropyl myristate with respect to the total weight of an oily phase.

19. The cosmetic according to claim 2, which is substantially free from cyclopentasiloxane, preferably totally free from cyclopentasiloxane.

20. The cosmetic according to claim 3, which is substantially free from cyclopentasiloxane, preferably totally free from cyclopentasiloxane.

21. The cosmetic according to claim 4, which is substantially free from cyclopentasiloxane, preferably totally free from cyclopentasiloxane.
